SEQUENCE LISTING (32 sequences)

SEQ ID NO: 1 - DNA, 72 bp, Artificial
  rfaJ deletion F
ctttaaacgtaaacttcttgaataaaacccataggtgatgtaatggattaaattaaccctcactaaagggcg

SEQ ID NO: 2 - DNA, 73 bp, Artificial
  rfaJ deletion R
agtttttaatctttttttcaataatcataatagagatttaggcaggggaataatacgactcactatagggctc

SEQ ID NO: 3 - DNA, 72 bp, Artificial
  phoP/phoQ deletion F
caacgctagactgttcttattgttaacacaagggagaagagatgatgcgcaattaaccctcactaaagggcg

SEQ ID NO: 4 - DNA, 73 bp, Artificial
  phoP/phoQ deletion R
ataacggatgcttaacgagatgcgtggaagaacgcacagaaatgtttatttaatacgactcactatagggctc

SEQ ID NO: 5 - DNA, 72 bp, Artificial
  lpxL deletion F
caaaaagatgcgagaatacggggaattgttcgttgaaagacaggatagaaaattaaccctcactaaagggcg

SEQ ID NO: 6 - DNA, 73 bp, Artificial
  lpxL deletion R
aaagctaaaagaggggaaaaattgcagcctgacggctgcaatcctgtcaataatacgactcactatagggctc

SEQ ID NO: 7 - DNA, 72 bp, Artificial
  lpxM deletion F
gacgtcgctacactattcacaattccttttcgcgtcagcagaccctggaaaattaaccctcactaaagggcg

SEQ ID NO: 8 - DNA, 73 bp, Artificial
  lpxM deletion R
catcaggtagtacagggtttgtcagcataaagcctctcttacgagaggcttaatacgactcactatagggctc

SEQ ID NO: 9 - DNA, 72 bp, Artificial
  pagP deletion F
tattcaggttaatgttgttattatcacagtcgaatttttgaacggtatgtaattaaccctcactaaagggcg

SEQ ID NO: 10 - DNA, 73 bp, Artificial
  pagP deletion R
ggctttttaattcacaacagaacaatgcccttctccgtcaaaactggaaataatacgactcactatagggctc

SEQ ID NO: 11 - DNA, 20 bp, Artificial
  phoP/phoQ F
ctgtttatccccaaagcacc

SEQ ID NO: 12 - DNA, 20 bp, Artificial
  phoP/phoQ R
gcgagagcggatcaataaag

SEQ ID NO: 13 - DNA, 20 bp, Artificial
  lpxL F
gctcaacgcaaaaagatgcg

SEQ ID NO: 14 - DNA, 20 bp, Artificial
  lpxL R
agggtgacatagcgttccac

SEQ ID NO: 15 - DNA, 20 bp, Artificial
  lpxM F
cgattaacaaatgcgctgac

SEQ ID NO: 16 - DNA, 20 bp, Artificial
  lpxM R
gttcaaccaataccacgcgt

SEQ ID NO: 17 - DNA, 20 bp, Artificial
  pagP F
cgccgttaacccgatactct

SEQ ID NO: 18 - DNA, 20 bp, Artificial
  pagP R
gctgtgtcggataccagtac

SEQ ID NO: 19 - DNA, 20 bp, Artificial
  rfaJ F
tccagtcgatgctgatactg

SEQ ID NO: 20 - DNA, 20 bp, Artificial
  rfaJ R
gtaaacccttctcgccgaac

SEQ ID NO: 21 - DNA, 19 bp, Artificial
  TNF-alpha F
cccctaccctgtcccacaa

SEQ ID NO: 22 - DNA, 21 bp, Artificial
  TNF-alpha R
tgagtactgcggagggttcat

SEQ ID NO: 23 - DNA, 22 bp, Artificial
  GAPDH F
ccccaatgtctctgttgttgac

SEQ ID NO: 24 - DNA, 22 bp, Artificial
  GAPDH R
cagccttcactaccctcttgat

SEQ ID NO: 25 - DNA, 24 bp, Artificial
  IL-1beta F
gctctacatgtcgtgtgtgatgag

SEQ ID NO: 26 - DNA, 18 bp, Artificial
  IL-1beta R
tgtcgatgtcccgcatga

SEQ ID NO: 27 - DNA, 20 bp, Artificial
  iNOS F
gcattcttattggcccagga

SEQ ID NO: 28 - DNA, 20 bp, Artificial
  iNOS R
catagagacgctgctgccag

SEQ ID NO: 29 - DNA, 20 bp, Artificial
  IL-18 F
acgtggcagcttttgaagat

SEQ ID NO: 30 - DNA, 20 bp, Artificial
  IL-18 R
gcggtggttttgtaacagtg

SEQ ID NO: 31 - DNA, 20 bp, Artificial
  TLR-4 F
ggcaaaaaatggaatcacga

SEQ ID NO: 32 - DNA, 20 bp, Artificial
  TLR-4 R
ctggaggaaggcaatcatca

(19) United States
(12) Patent Application Publication
(10) Pub. No.: US 2022/0395565 A1
(43) Pub. Date: Dec. 15, 2022

(54) ATTENUATED SALMONELLA GALLINARUM MUTANT STRAINS AND USES THEREOF

(71) Applicant: SEOUL NATIONAL UNIVERSITY R&DB FOUNDATION, Seoul (KR)

(72) Inventors: Hyuk-Joon KWON, Gangwon-do (KR); Nam-Hyung KIM, Gyeonggi-do (KR); Dae-Sung KO, Gyeonggi-do (KR); Eun-Jin HA, Seoul (KR); Sunmin AHN, Seoul (KR)

(21) Appl. No.: 17/824,452

(22) Filed: May 25, 2022

(30) Foreign Application Priority Data
May 26, 2021 (KR) 10-2021-0067910

(51) Int. Cl. A61K 39/112; C12N 1/20; C12N 15/90; A61P 31/04

(52) U.S. Cl. CPC A61K 39/0275; C12N 1/205; C12N 15/902; A61P 31/04; C12R 2001/42

(57) ABSTRACT

The present disclosure relates to Salmonella Gallinarum mutant strains and uses thereof. A vaccine composition according to an aspect has no risk of reverting to pathogenicity, has no residual pathogenicity due to detoxification of an endotoxin, and does not cause lesions and bacterial re-isolation, thereby exhibiting significantly improved safety compared to the existing fowl typhoid vaccines. In addition, since the vaccine composition induces a high-level immune response even when administered to young chicks, it may be used regardless of age, and as the vaccine strain may be used as a live vaccine having an excellent protective capability by itself, the vaccine composition may be useful for preventing and alleviating fowl typhoid.

### CROSS-REFERENCE TO RELATED APPLICATION

This application claims the benefit of Korean Patent Application No. 10-2021-0067910, filed on May 26, 2021, in the Korean Intellectual Property Office, the disclosure of which is incorporated herein in its entirety by reference.

### BACKGROUND

### 1. Field

The present disclosure relates to Salmonella Gallinarum mutant strains and uses thereof.

### 2. Description of the Related Art

Fowl typhoid is a major bacterial disease caused by Salmonella Gallinarum (Salmonella enterica subsp. enterica serovar Gallinarum biovar Gallinarum, S. Gallinarum), which is morphologically a Gram-negative, short rod bacterium, and refers to an acute or chronic infectious disease that occurs in birds such as chickens and turkeys. The disease may occur at any age, and its mortality rate is high due to sepsis. Common symptoms of fowl typhoid infection in chickens are include acute death, enlargement of the liver, spleen, and kidneys, white necrotic spots in the liver and bronze liver, watery or mucous yellow diarrhea, and reduction in egg laying, and a typhus outbreak during an egg-laying period may lead to sporadic deaths over a long period of time. The incidence of fowl typhoid in Korea increased gradually from the first report in 1992 until 2001, and has continued to grow even until recently with numbers of incidence of 121,495 in 2017, 112,009 in 2018, and 101,204 in 2019. Given that fowl typhoid in a mature chicken or a white semi broiler does not cause severe symptoms, and is treated with administration of antibiotics on site, the actual incidence is expected to be higher than reported and lead to enormous economic damage.

Several vaccines have been developed for the prevention of fowl typhoid, but the live attenuated SG9R vaccine is currently the most popular product on the market. Live vaccines are known to be stronger and longer-lasting than killed or inactivated vaccines because the vaccine strain activates the immune system by going through an infection path similar to that of field isolates. The SG9R strain is attenuated by a nonsense mutation of an adenine in position 9 to a cytosine in an rfaJ gene, which is one of the genes involved in the synthesis of lipopolysaccharides, a key antigen of Gram-negative bacteria. Mutation of the rfaJ gene causes a phenotypic change to a semi-rough strain having short polysaccharide chains in LPS due to failure of all subsequent polysaccharide synthesis. The short polysaccharide chains reduce resistance to the immune system of the host, such as antibodies and complements, weaken pathogenicity, and thus, enables the SG9R strain to be used as a vaccine. However, as the SG9R strain is attenuated by a mutation of a single base, there is a possibility of acquisition of pathogenicity due to a back mutation, and in fact, a number of cases have been reported in which bacteria with a genetic background similar to that of the vaccine strain were isolated from farms having previous vaccination with SG9R. Moreover, SG9R also has strong potential pathogenicity of its own and may cause liver lesions or even death in severe cases in young chicks, or in chickens immunocompromised due to malnutrition or other diseases. Therefore, there is a need for a novel fowl typhoid vaccine with improved safety as compared to the existing vaccine strains.

### SUMMARY

The present inventors developed a Salmonella Gallinarum mutant strain Safe-9R, in which an rfaJ gene in SG9R is artificially deleted, and Salmonella Gallinarum mutant strains Dtx-9RL and Dtx-9RM, in which an IpxL gene and an IpxM gene are additionally deleted from the Safe-9R, respectively, and found that the attenuated Salmonella Gallinarum strains exhibit sufficient immunity when inoculated in young chicks, in particular, and have low potential pathogenicity without any risk of reverting to pathogenicity and the endotoxin detoxified, thereby completing the present invention.

An aspect is to provide a Salmonella Gallinarum strain in which an rfaJ gene is deleted.

Another aspect is to provide a vaccine composition for preventing fowl typhoid comprising the Salmonella Gallinarum strain as an active ingredient.

Still another aspect is to provide a feed composition for preventing fowl typhoid comprising the Salmonella Gallinarum strain as an active ingredient.

### BRIEF DESCRIPTION OF THE DRAWINGS

FIG. 1 shows PCR results confirming deletion of the rfaJ gene in Safe-9R in which the rfaJ gene was deleted from Salmonella Gallinarum and knock-out mutant strains derived from Safe-9R. (A) Confirmation of deletion of the rfaJ gene from Safe-9R and Safe-9R-derived knock-out strains. Lane 1: SG9R; Lane 2: Safe-9R, Lane 3: ΔphoP/Q; Lane 4: ΔIpxL; Lane 5: ΔIpxM, Lane 6: ΔpagP. (B) Confirmation of deletion of lipid A biosynthesis-related genes in Safe-9R-derived knock-out strains. The amplicon of Safe-9R for each gene was placed at the first lane of each rectangle to compare with the amplicon of the knock-out mutant strain.

FIGS. 2A to 2C are diagrams showing sequencing results confirming deletion of the rfaJ gene in Salmonella Gallinarum.

FIG. 3 shows results of humoral immune responses of OE (oil emulsion) Safe-9R vaccine strains. One-week-old chicks were inoculated with Safe-9R vaccine strains, and serum samples were collected at 2 weeks post-vaccination (wpv) and 7 wpv, respectively. The immune response was analyzed by ELISA made by Salmonella enterica serovar Gallinarum biovar Gallinarum (SG) immunogenic outermembrane proteins (OMP), OmpA and OmpX, and total OMP extracts (*p<0.05).

FIG. 4 shows charts comparing effects of each knock-out mutant strain on transcriptions of pro-inflammatory cytokines and related genes in HD11 cells. Relative transcription levels of IL-1β, IL-18, iNOS and TLR4 genes were compared by using the 2-ΔΔCt method, after infecting HD11 cells with each knock-out mutant strain (ns: statistically not significant, *p<0.05).

FIG. 5 shows results verifying the toxicity of the endotoxin-detoxified mutant strains by examining the body weight increase. (A) One-week-old and (B) Two-week-old chicks were inoculated with inactivated vaccines, and the differences in body weight were examined (*p<0.05).

FIG. 6 shows charts confirming humoral immunogenicity of endotoxin-detoxified mutant strains inoculated as oil emulsions (*p<0.05).

FIG. 7 shows results confirming humoral and mucosal immunogenicity of live endotoxin-detoxified mutant strains (*p<0.05).

FIG. 8 shows the proportion of CD8+ T cells in peripheral blood mononuclear cells (PBMCs) analyzed by fluorescence activated cell sorting (FACS).

FIG. 9 shows results of removing an antibiotics resistance gene from vaccine candidate strains by using the flippase-flippase recognition target (FLP-FRT) recombination system. Lanes 1, 2: Dtx9RMΔkana; Lane 3: Dtx9RM; Lane 4: Safe9R; Lane 5: Negative control group. The primer used targets the IpxM gene.

### DETAILED DESCRIPTION

Reference will now be made in detail to embodiments, embodiments of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

An aspect provides a Salmonella Gallinarum strain in which an rfaJ gene is knocked out.

The rfaJ gene is a gene encoding a lipopolysaccharide 1,2-glucosyltransferase, and, for example, may be a base sequence corresponding to 3904235 to 3905248 in a base sequence of a genome corresponding to NCBI accession number: NC_011274.1 (https://www.ncbi.nlm.nih.gov/nuccore/NC_011274.1?report=fasta&from=3904235&to=3905248).

The Salmonella Gallinarum mutant strain according to an aspect is characterized by having the rfaJ gene itself deleted, rather than having a single nucleotide change in the rfaJ.

As used herein, the term “deletion” refers to a partial, substantial, or complete knock-out, silencing, inactivation or down-regulation of a gene. Any method known in the art to delete a gene may be used without limitation, and for example, a method of deleting a gene by using a homologous recombination may be used.

As used herein, the term “homologous recombination” refers to a type of genetic recombination that occurs through the connection and exchange at loci of genetic sequences homologous to each other.

In an embodiment, the rfaJ gene was deleted from SG9R via a homologous recombination to obtain a Salmonella Gallinarum mutant strain in which the rfaJ gene is deleted, which is named Safe-9R.

The present specification also provides a Salmonella Gallinarum strain, in which an IpxL gene is additionally deleted from the Safe-9R.

The present specification also provides a Salmonella Gallinarum strain, in which an IpxM gene is additionally deleted from the Safe-9R.

The IpxL gene is a gene encoding a lipid A biosynthesis lauroyltransferase and, for example, may be a base sequence corresponding to 2027901 to 2028821 in a base sequence of a genome corresponding to NCBI accession number: NC_011274.1 (<https://www.ncbi.nlm.nih.gov/nuccore/NC_011274.1?report=fasta&from=2027901&to=2028821>).

The IpxM gene is a gene encoding a lipid A biosynthesis myristoyltransferase and, for example, may be a base sequence corresponding to 1245903 to 1246874 in a base sequence of a genome corresponding to NCBI accession number: NC_011274.1 (https://www.ncbi.nlm.nih.gov/nuccore/NC_011274.1?report=fasta&from=1245903&to=1246874).

In an embodiment, the IpxL or IpxM gene was additionally removed by using a homologous recombination, and the Salmonella Gallinarum mutant strain, in which the IpxL or the IpxM gene is additionally deleted is named Dtx-9RL and Dtx-9RM, respectively.

The Safe-9R, Dtx-9RL and Dtx-9RM mutant strains may be ones in which an antibiotic resistance gene is further deleted. The antibiotic resistance gene may be a tetracycline resistance gene or a kanamycin resistance gene, but is not limited thereto.

Another aspect provides a vaccine composition for preventing fowl typhoid comprising the above-described Salmonella Gallinarum strain (SAFE-9R, Dtx-9RL, Dtx-9RM mutant strain; or a mutant strain in which an antibiotic resistance gene is further deleted) as an active ingredient.

As used herein, the term, “prevention” refers to all acts of inhibiting or delaying infection of fowl typhoid by administrating the vaccine composition according to an aspect.

As used herein, the term, “vaccine” refers to a biological formulation containing an antigen that provides immunity thereto in a living body, and refers to an immunogen or an antigenic substance that generates immunity in a living body by injection or oral administration to a human or animal subject to prevent infection.

Types of vaccines include attenuated vaccines in which weakened pathogenicity, and inactivated vaccines or killed vaccines in which pathogens are completely killed. The attenuated vaccines are also known as live pathogen vaccines, and the inactivated vaccines are known as killed vaccines or a toxoid vaccines.

As used herein, the term, “live vaccine” may be used to include attenuated vaccines or live vaccines, and the live vaccine refers to bacteria or virus having weakened pathogenicity, causing no or reduced clinical symptoms, when administered to an animal. The attenuated Salmonella Gallinarum may be isolated by using a method known in the art, for example, by subculturing a virus.

As used herein, the term, “killed vaccine” may be used to include inactivated vaccines or killed vaccines, and the killed vaccine refers to bacteria or virus heated or treated with chemicals such as formalin or phenol to inactivate pathogenicity, while retaining immunogenicity. The inactivation may be performed by using a method known in the art without a limitation, and preferably may be performed by heating the live attenuated Salmonella Gallinarum.

A vaccine composition according to an aspect may be used as a live vaccine, or a killed vaccine of the attenuated Salmonella Gallinarum. In particular, in an embodiment, the vaccine composition according to an aspect was found to have excellent safety and protective efficacy compared to existing vaccines when inoculated in young chicks in a form of a live vaccine, and was confirmed to be useful as a vaccine composition for preventing fowl typhoid.

A dose of the vaccine composition may vary depending on the body weight, age, sex, health condition, nutrition, and excretion rate of the chicken, duration and route of administration, and the severity of the disease, and the vaccine composition may be administered in a single dose or multiple doses.

The vaccine composition may be administered via at least one route selected from the group consisting of oral, percutaneous, intramuscular, intraperitoneal, intradermal, subcutaneous and intranasal routes, and preferably may be administered via an intramuscular route, but is not limited thereto.

The vaccine composition may further include at least one selected from the group consisting of a carrier, a diluent, and an adjuvant.

The carrier may be a veterinarily acceptable carrier. As used herein, the term “veterinarily acceptable carrier” includes any and all solvents, dispersion mediums, coating agents, antigen adjuvants, stabilizers, diluent, preservatives, antibacterial and antifungal agents, isotonic agents, adsorption delaying agents, and the like. A carrier, an excipient, or a diluent that may be included in the vaccine composition include lactose, dextrose, sucrose, sorbitol, mannitol, xylitol, maltitol, starch, glycerin, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, mineral oil, and the like.

In addition, the vaccine composition may be formulated and used as oral formulations such as powders, granules, tablets, capsules, suspensions, emulsions, syrups and aerosol, nasal formulations such as drips or sprays, and sterilized solutions for injection. The formulation may be prepared by using a diluent or an excipient such as a filler, a thickening agent, a binder, a wetting agent, a disintegrant, or a surfactant. Solid formulations for oral administration include tablets, pills, powders, granules, capsules, and the like, and these solid formulations may be prepared by mixing lecithin-like emulsifier with at least one excipient such as starch, calcium carbonate, sucrose or lactose, gelatin.

Also, lubricants such as magnesium stearate, or talc may be further included in addition to simple excipients. Liquid formulations for oral administration include suspensions, oral liquids, emulsifiers, syrups, and the like, and various excipients, for example, a wetting agent, a sweetener, a fragrance, or a preservative may be included in addition to commonly used simple diluents such as water, or liquid paraffin. Formulations for parenteral administration include sterile aqueous solutions, non-aqueous solvents, suspensions, emulsions, and lyophilized preparations. As the non-aqueous solvent and the suspension, propylene glycol, polyethylene glycol, a vegetable oil such as olive oil, an injectable ester such as ethyl oleate may be used, but is not limited thereto. Penetrants suitable for formulations for nasal administration are generally known to those skilled in the art. Such suitable formulations are preferably formulated to be sterile, isotonic, and buffered, for safety and compliance. In addition, formulations for nasal administration are formulated to stimulate mucus secretion in several ways to maintain normal ciliary function, and as described in a reference (Remington's Pharmaceutical Science, 18th Ed., Mack Publishing Co., Easton, Pa. (1990)), are preferably isotonic, slightly buffered formulations maintaining a pH of 5.5 to 6.5, and most preferably, include antimicrobial preservatives and suitable drug stabilizers.

In addition, the vaccine composition may further contain at least one second adjuvant selected from the group consisting of stabilizers, emulsifiers, aluminum hydroxide, aluminum phosphate, pH adjusters, surfactants, liposomes, iscom adjuvants, synthetic glycopeptides, extenders, carboxypolymethylene, subviral particle adjuvant, cholera toxin, N,N-dioctadecyl-N′,N′-bis(2-hydroxyethyl)-propanediamine, monophosphoryl lipid A, dimethyldioctadecyl-ammonium bromide, Marcol-Aracel, chlorhexidine, and a combination thereof. Preferably, MONTANIDE ISA70 (VG), ISA71 (VG), ISA71R (VG), ISA206 (VG), ISA201 (VG), ISA763 (A VG), IMS1313 (VG N) or GEL01 may be used, and most preferably, MONTANIDE ISA70 (VG) may be used as an adjuvent.

Still another aspect provides a feed composition for preventing fowl typhoid comprising the above-described Salmonella Gallinarum strain as an active ingredient.

The feed composition has excellent protective efficacy against fowl typhoid and may contribute to prevention of and immunity enhancement against fowl typhoid.

The feed composition may use live, or killed pathogens of the Salmonella Gallinarum mutant strain according to an aspect alone, or in combination with carriers, stabilizers, and the like known in the art, such as grains and by-products thereof allowed for poultry farming, and further include organic acids such as citric acid, humic acid, adipic acid, lactic acid, and malic acid, phosphates such as sodium phosphate, potassium phosphate, acid pyrophosphate and polyphosphate, natural antioxidants such as polyphenols, catechins, alpha-tocopherol, rosemary extract, vitamin C, green tea extract, licorice extract, chitosan, tannic acid, and phytic acid, antibiotics, antibacterial agents and other additives. The feed composition may be in any suitable form such as powders, granules, pellets, suspensions, and the like, and may be supplied alone or as a mixture with other feed for poultry.

Another aspect provides a method of preventing fowl typhoid comprising inoculating to a subject of poultry the vaccine composition according to an aspect.

In a specific example, the poultry may be one selected from the group consisting of chickens, ducks, turkeys, geese, quails, pheasants and wild geese, but is not limited thereto.

For example, the poultry may be chicken, and a chick less than 2 weeks old, but is not limited thereto.

The vaccine composition according to an aspect has no risk of reverting to pathogenicity, has no residual pathogenicity due to detoxification of the endotoxin, and does not cause lesions and bacterial re-isolation, thereby exhibiting a significantly improved safety compared to the existing fowl typhoid vaccines. In addition, since the vaccine composition induces a high-level immune response even when administered to young chicks, it may be used regardless of the age, and as the vaccine strain may be used as a live vaccine having an excellent protective efficacy by itself, the vaccine composition may be useful for preventing and alleviating fowl typhoid.

### EXAMPLES

Hereinafter, the present disclosure will be described in more detail through examples. However, these examples are intended to illustrate at least one specific example, and the scope of the present disclosure is not limited to these examples.

### Example 1. Generation of Improved Vaccine Strain Through Gene Deletion

1-1. Deletion of rfaJ Gene

Gene deletion or knockout was performed by using the Gene Bridges (Germany)′ Red/ET recombination kit (catalog number: K006). The kit may delete a gene by applying the Lambda-Red technology that breaks the double-stranded DNA at a targeted site, and repairing the damaged gene via a homologous recombination.

First, for the gene deletion, a homology arm for the region to be deleted was produced by using PCR. The region to be deleted from the rfaJ gene is from 279 nt to 1284 nt, which corresponds to the region from the position of the nonsense mutation in SG9R to the stop codon in the gene. An attenuated Salmonella Gallinarum vaccine strain SNU5161 was cultured in Luria Bertani (LB) broth (Difco, USA) at 37° C. for 16 hours. SNU5161 was isolated from the liver and feces of poultry in Korea which had been inoculated with an attenuated live SG9R vaccine for fowl typhoid, and was characterized to be genetically identical to SG9R. The cultured bacteria were washed three times with distilled water containing 10% glycerol to promote transformation. The prepared bacteria were transformed with a Red/ET expression plasmid for a recombinant protein from the above-described kit by electroporation (2,000 V, 10 μF, 600 Ohms). Next, the bacteria were cultured with shaking in 1 ml of LB broth at 30° C. for 70 minutes, and incubated on LB agar with tetracyclin at 30° C. for at least 16 hours.

The colony identified on the agar was cultured with shaking at 30° C. in tetracycline-added LB broth, and then cultured with shaking at 37° C. for an hour after adding 50 μl of 10% L-arabinose. Next, the cultured bacteria were washed three times with distilled water containing 10% glycerol, and were transformed with the homology arms by electroporation in the same manner as described above. Next, the transformed bacteria were cultured with shaking in LB broth at 37° C. for 3 hours, and then cultured without shaking for at least 16 hours on LB agar with kanamycin. The generated colony was found by PCR to have a kanamycin-added cassette inserted in place of the pre-existing gene.

Next, in order to remove kanamycin resistance from the vaccine strain, the bacteria in which the rfaJ gene was deleted was transformed with 1 μl of a 707-FLPe plasmid by electroporation, and cultured with shaking in LB broth at 30° C., for 70 minutes. Next, a strain without antibiotic resistance was selected by culturing the bacteria both on kanamycin-added LB agar and general LB agar.

1-2. Deletion or Knockout of IpxL and IpxM Genes

In the same manner as described above, IpxL or IpxM genes was additionally deleted from the mutant strain prepared in Example 1-1, in which the rfaJ gene is deleted. The IpxL and IpxM genes are involved in lipid A biosynthesis along with pagP, phoP/phoQ (phoP/Q) genes, and the lipid A constitutes an endotoxin of a Gram-negative bacteria such as Salmonella. For the IpxL gene, the region from the start codon to 917 nt was deleted, and for the IpxM gene, the region from the start codon to the stop codon was deleted.

1-3. Confirmation of Generated Strains

The mutant strains generated in Examples 1-1 and 1-2 were confirmed via PCR and sequencing. Specifically, bacterial genomic DNA was extracted by using the G-spin Genomic DNA Extraction Kit for Bacteria (iNtRON Biotechnology, Korea) and PCR was conducted using 1 μL of the template DNA (50 ng/μL), 3 μL of 10× buffer, 3 μL of dNTPs (5 mM), 0.5 μL of each primer (10 pmol/μL), and 0.25 μL of Taq polymerase (MGmed, Korea) under the following conditions: 95° C. for 5 minutes; followed by 35 cycles of 95° C. for 30 seconds, 55° C. for 30 seconds, and 72° C. for 1 minute; and lastly, 72° C. for 5 minutes. PCR amplicons were purified by using the PCR/Gel Purification Kit (MGmed), and sequencing was performed by using the ABI 3711 automatic sequencer (Cosmogenetech, Korea). Nucleotide sequences were translated and compared by using the BioEdit program version 7.2.5. The extracted genomic DNA was sequenced by using the HiSeq 2000 platform (Illumina, San Diego, Calif., USA) and the filtered data were mapped by using the BWA version 0.7.12 to S. enterica serovar Gallinarum str. 287/91 (GenBank Accession Number NC_011274.1) in the National Center for Biotechnology Information database to identify differences between Safe-9R and SG9R. All primers used in the experiments are described in Table 1.

TABLE 1

| | | SEQ |
|---|---|---|
| | | ID |
| Primer | Sequence (5′-3′) | NO. |
| rfaJ | CTTTAAACGTAAACTTCTTGAATAAAACCCATAGGT | 1 |
| dele- | GATGTAATGGATTAAATTAACCCTCACTAAAGGGCG | |
| tion F | | |
| rfaJ | AGTTTTTAATCTTTTTTTCAATAATCATAATAGAGA | 2 |
| dele- | TTTAGGCAGGGGAATAATACGACTCACTATAGGGCT | |
| tion R | C | |
| phoP/ | CAACGCTAGACTGTTCTTATTGTTAACACAAGGGAG | 3 |
| phoQ | AAGAGATGATGCGCAATTAACCCTCACTAAAGGGCG | |
| dele- | | |
| tion F | | |
| phoP/ | ATAACGGATGCTTAACGAGATGCGTGGAAGAACGCA | 4 |
| phoQ | CAGAAATGTTTATTTAATACGACTCACTATAGGGCT | |
| dele- | C | |
| tion R | | |
| lpxL | CAAAAAGATGCGAGAATACGGGGAATTGTTCGTTGA | 5 |
| dele- | AAGACAGGATAGAAAATTAACCCTCACTAAAGGGCG | |
| tion F | | |
| lpxL | AAAGCTAAAAGAGGGGAAAAATTGCAGCCTGACGGC | 6 |
| dele- | TGCAATCCTGTCAATAATACGACTCACTATAGGGCT | |
| tion R | C | |
| lpxM | GACGTCGCTACACTATTCACAATTCCTTTTCGCGTC | 7 |
| dele- | AGCAGACCCTGGAAAATTAACCCTCACTAAAGGGCG | |
| tion F | | |
| lpxM | CATCAGGTAGTACAGGGTTTGTCAGCATAAAGCCTC | 8 |
| dele- | TCTTACGAGAGGCTTAATACGACTCACTATAGGGCT | |
| tion R | C | |
| pagP | TATTCAGGTTAATGTTGTTATTATCACAGTCGAATT | 9 |
| dele- | TTTGAACGGTATGTAATTAACCCTCACTAAAGGGCG | |
| tion F | | |
| pagP | GGCTTTTTAATTCACAACAGAACAATGCCCTTCTCC | 10 |
| dele- | GTCAAAACTGGAAATAATACGACTCACTATAGGGCT | |
| tion R | C | |
| phoP/ | CTGTTTATCCCCAAAGCACC | 11 |
| phoQ F | | |
| phoP/ | GCGAGAGCGGATCAATAAAG | 12 |
| phoQ R | | |
| lpxL F | GCTCAACGCAAAAAGATGCG | 13 |
| lpxL R | AGGGTGACATAGCGTTCCAC | 14 |
| lpxM F | CGATTAACAAATGCGCTGAC | 15 |
| lpxM R | GTTCAACCAATACCACGCGT | 16 |
| pagP F | CGCCGTTAACCCGATACTCT | 17 |
| pagP R | GCTGTGTCGGATACCAGTAC | 18 |
| rfaJ F | TCCAGTCGATGCTGATACTG | 19 |
| rfaJ R | GTAAACCCTTCTCGCCGAAC | 20 |
| TNF-α F | CCCCTACCCTGTCCCACAA | 21 |
| TNF-α R | TGAGTACTGCGGAGGGTTCAT | 22 |
| GAPDH F | CCCCAATGTCTCTGTTGTTGAC | 23 |
| GAPDH R | CAGCCTTCACTACCCTCTTGAT | 24 |
| IL-1β F | GCTCTACATGTCGTGTGTGATGAG | 25 |
| IL-1β R | TGTCGATGTCCCGCATGA | 26 |
| iNOS F | GCATTCTTATTGGCCCAGGA | 27 |
| iNOS R | CATAGAGACGCTGCTGCCAG | 28 |
| IL-18 F | ACGTGGCAGCTTTTGAAGAT | 29 |
| IL-18 R | GCGGTGGTTTTGTAACAGTG | 30 |
| TLR-4 F | GGCAAAAAATGGAATCACGA | 31 |
| TLR-4 R | CTGGAGGAAGGCAATCATCA | 32 |

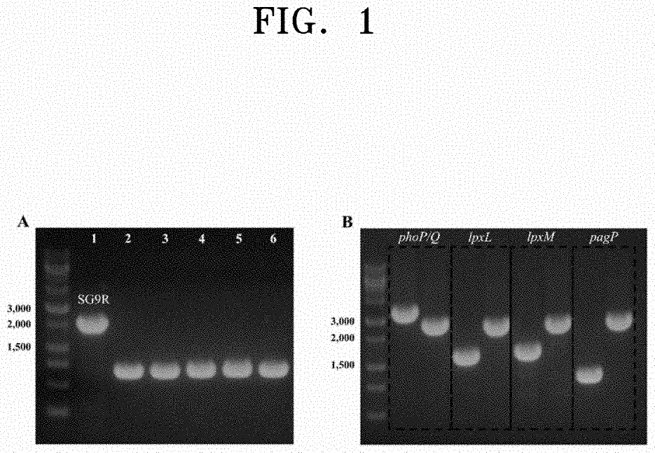

As a result, as shown in FIG. 1, the sequencing results confirmed that the rfaJ gene was successfully removed, and the rfaJ gene was found to be removed from all the mutant strains. The mutant strain, in which the rfaJ gene was deleted, was named Safe-9R, and the knock-out mutant strains, in which the IpxL or IpxM gene was additionally deleted was named Dtx-9RL and Dtx-9RM, respectively.

The Safe-9R strain was deposited to the Korean Collection for Type Cultures of Korea Research Institute of Bioscience and Biotechnology on Feb. 4, 2021, and was given the accession number of KCTC14464BP. The Dtx-9RL strain was deposited to the Korean Collection for Type Cultures of Korea Research Institute of Bioscience and Biotechnology on Feb. 4, 2021, and was given the accession number of KCTC14463BP. The Dtx-9RM strain was deposited to the Korean Collection for Type Cultures of Korea Research Institute of Bioscience and Biotechnology on May 21, 2020, and was given the accession number of KCTC18822P.

### Example 2. Analysis of Base Sequence and Homology of Safe-9R and SG9R

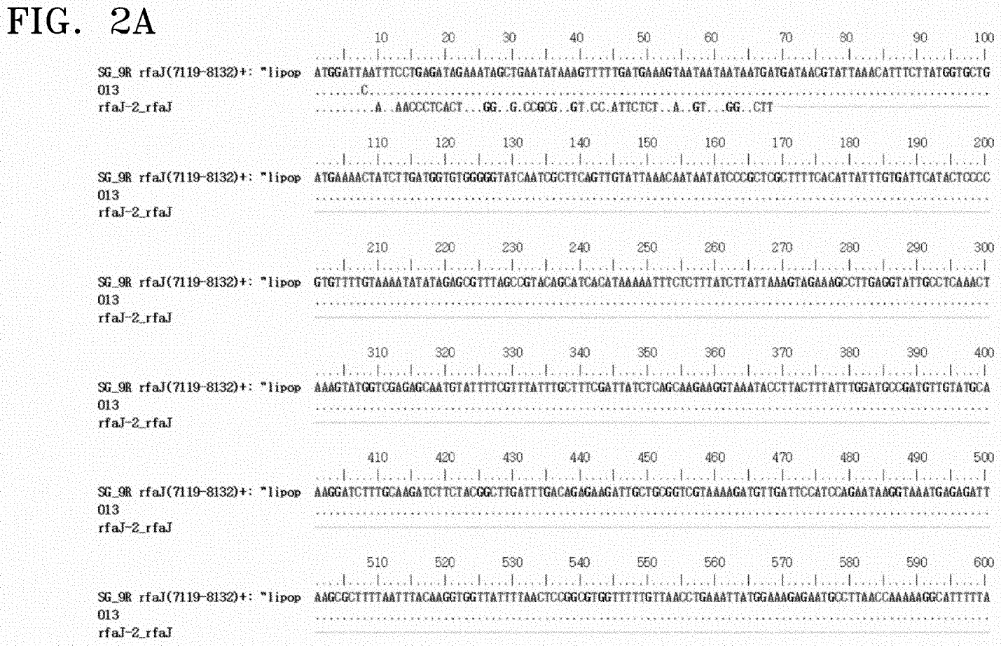

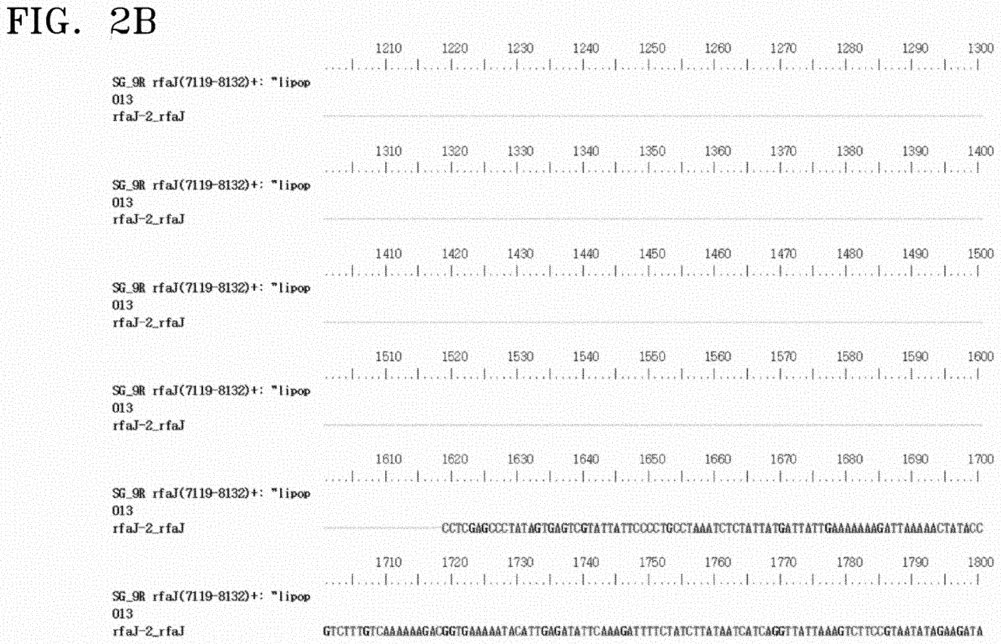

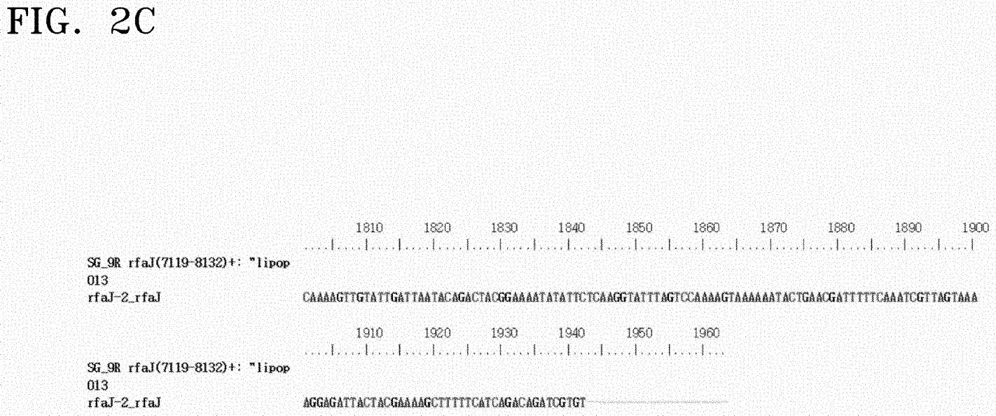

The genomic DNA of Safe-9R prepared in Example 1 was extracted and compared with the base sequence of SG9R (SNU5161), is the parent strain (FIG. 2). Upon comparing Safe-9R and SG9R by the next generation sequencing (NGS) setting NC_011274.1 registered in National Center for Biotechnology Information (NCBI) as the reference SG9R strain, 6 genetic differences were identified including the rfaJ gene, but one was not in the coding region, and three were silent mutations. The last one was a missense mutation in which threonine was mutated to serine, but threonine and serine are almost similar except for the difference between a methyl group and a hydrogen ion, and the protein was not related to pathogenicity. Hence, no significant phenotypic difference was expected.

### Example 3. Biochemical Properties of Safe-9R

Biochemical properties of Safe-9R prepared in Example 1 were identified by using a VITEK device, and the results are shown in Table 2 below. As shown in Table 2, Safe-9R was negative to glucose/Fer, maltose, coumarate, 0129 resistance (comp vibrio), while a pathogenic fowl typhoid bacteria or SG9R vaccine strain were shown to be positive thereto. Thus, Safe-9R was found to be significantly different from them in biochemical properties.

TABLE 2

| | | SG | SG9R (existing |
|---|---|---|---|
| GN Card | Safe-9R | (field strain) | vaccine strain) |
| 1. adonitol | − | − | − |
| 2. cellobiose | − | − | − |
| 3. H2S | − | − | − |
| 4. D-glucose | + | + | + |
| 5. glucose/Fer | | + | + |
| 6. D-maltose | − | + | + |
| 7. D-mannitol | + | + | + |
| 8. D-mannose | + | + | + |
| 9. lipase | − | − | − |
| 10. Urease | − | − | − |
| 11. sorbitol | − | − | − |
| 12. sucrose | − | − | − |
| 13. trehalose | + | + | + |
| 14. citrate | − | − | + |
| 15. malonate | − | − | − |
| 16. phosphatate | (−) | − | (+) |
| 17. glycine | − | − | − |
| 18. ornithine | − | − | − |
| 19. lysine | + | + | + |
| 20. decarboxylase | | | |
| 21. histidine | − | − | − |
| 22. coumarate | − | + | + |
| 23. O129 resistance | − | + | + |
| (comp vibrio) | | | |

### Example 4. Confirmation of Genetic Stability of Safe-9R

The genetic stability of Safe-9R prepared in Example 1 was confirmed by performing blind passages. Specifically, Safe-9R was cultured with shaking in LB broth at 37° C. for 24 hours, then 1 ml of the culture was passed to a fresh LB broth and cultured under the same condition, and the cycle was repeated 10 times in the same way. Then, it was examined by PCR and sequencing whether a mutation occurred in the deleted region in the rfaJ gene. As a result, Safe-9R was found to be genetically very stable without any mutation in the rfaJ gene after 10 passages.

### Example 5. Experiments on Vaccine Efficacy of Safe-9R

Vaccine efficacy and toxicity of Safe-9R were examined. Specifically, live Safe-9R vaccines were inoculated to brown layer chicks at 6 and 18 weeks at 1×107 colony forming units (cfu)/chicken, and 4 weeks after the second vaccination, the pathogenic field strain SG0197 (1×108 cfu/chicken) was challenged. The OE (oil emulsion) Safe-9R vaccine was prepared by heat treatment of Safe-9R at 65° C. for 2 hours, followed by gradual cooling to room temperature and emulsification of bacteria and oil adjuvant (Montanide ISA 70, France) at a ratio of 3:7. The OE killed Safe-9R vaccine was intramuscularly injected to 1-week old brown layer chicks at approximately 1×109 cfu/100 μL/chick, and 2 weeks and 7 weeks after the vaccination, SG0197 was challenged. Serum samples were collected before the challenge, and infection and mortality rate were observed for 17 days. The chickens used in the experiment were immunocompromised chickens fasted for three days (protein-energy malnutrition; PEM models). The results are shown in Table 3.

TABLE 3

| Group | Vaccination | Survival rate | | |
|---|---|---|---|---|
| Live Safe-9R | Vaccinated | 100 (10/10)* | | |
| vaccine | Unvaccinated | 0 (0/10) | | |
| OE killed | Vaccinated | 60 (6/10)* | 50 (5/10) | 80 (8/10) |
| Safe-9R | | | | |
| vaccine ^{a}—2 | Unvaccinated | 0 (0/10) | 11.1 (1/9) | 50 (5/10) |
| wpv ^{b} | | | | |
| OE killed | Vaccinated | 87.5 (7/8) | 50 (5/10) | 70 (7/10) |
| Safe-9R | | | | |
| vaccine ^{a}—7 | Unvaccinated | 80 (8/10) | 90 (9/10) | 90 (9/10) |
| wpv ^{b} | | | | |

a The Oil emulsion (OE) killed vaccine was inoculated to one-week-old, and challenged to three-week-old (2 wpv) and eight-week-old (7 wpv). All tests were performed in triplicates. b wpv refers to week post-vaccination.

*A significant difference compared to the control group

As shown in Table 3, live Safe-9R vaccines showed 100% protection efficacy against the challenge of the pathogenic field strain, resulting in no mortality in the vaccinated group, in contrast to the unvaccinated group. Upon examining efficacy of a killed Safe-9R vaccine, the OE killed vaccine was also found to show a significant difference when challenged at 2 wpv.

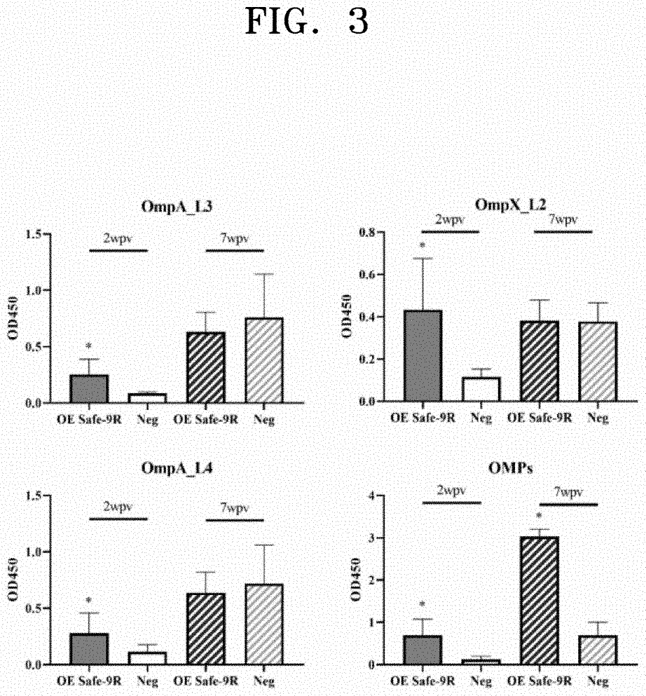

Next, humoral immune response of the OE Safe-9R killed vaccine was evaluated. Specifically, the vaccine strain was injected to 15 one-week-old brown layer chicks via the intramuscular route, and blood samples were collected at 2 wpv. Antibody titers of the OE killed vaccines were evaluated by using OmpA and OmpX peptide ELISA, and OMP (outer membrane protein) ELISA. As shown in FIG. 3, antibody titers of anti-OmpA and anti-OmpX were found to be significantly higher in the 2 wpv group compared to the unvaccinated group.

### Example 6. Experiment Comparing Toxicity of Endotoxin-Detoxified Mutant Strains

The toxicity of endotoxin-detoxified mutant strains Dtx-9RL and Dtx-9RM, and the existing vaccine strain SG9R was compared. In order to compare the capability to stimulate expression of pro-inflammatory cytokines in HD11, a chicken macrophage cell line, each strain was inoculated to the chicken macrophage (HD11) and reverse transcriptase quantitative-PCR (RT-qPCR) was performed. Specifically, the HD11 cell line was cultured using the RPMI 1640 medium (Thermo Fisher Scientific, Waltham, Mass., USA) modified with L-glutamine and phenol red and supplemented with 10% FBS (fetal bovine serum). Two days before the Salmonella infection, HD11 cell suspension (1×106 cells/mL) was seeded into each well of a 24-well plate at a volume of 500 μL/well and the cells were allowed to grow to approximately 85% confluence. The overnight culture of the bacteria was adjusted to an optical density of 0.2 at 600 nm and a ten-fold dilution was performed using phosphate-buffered saline (PBS) to obtain multiplicity of infection (MOI) of 10. The diluted bacterial suspension was centrifuged at 11,000 g for 1 min and resuspended in RPMI 1640. The cells were washed twice with the medium and inoculated with the bacterial suspension and incubated for 2 hours at 37° C. under a 5% CO2 condition. 2 hours after the infection, the cells were washed once and incubated in a medium supplemented with 150 μL/mL of gentamicin sulfate for another 2 hours. After washing the cells with the medium, total RNA was extracted by using the RNeasy mini kit (Qiagen, Germany). An amount of cDNA equal to the total RNA was synthesized by using the AmfiRivert cDNA Synthesis Platinum Master Mix (GenDEPOT, USA). RT qPCR was performed by using 10 μL of a reaction mixture including 5 μL of 2× AMPIGENE qPCR Green Mix Hi-ROX (Enzo Life Sciences, USA), 0.5 μL of forward primers, 0.5 μL of backward primers, and 1 μL of cDNA. The normalization was performed using glyceraldehyde 3-phosphate dehydrogenase (GAPDH), and all primers used in the RT-qPCR are listed in Table 1. Relative mRNA expression levels of IL-1β, IL-18, iNOS and TLR4 genes were compared by using the 2 −ΔΔCt method.

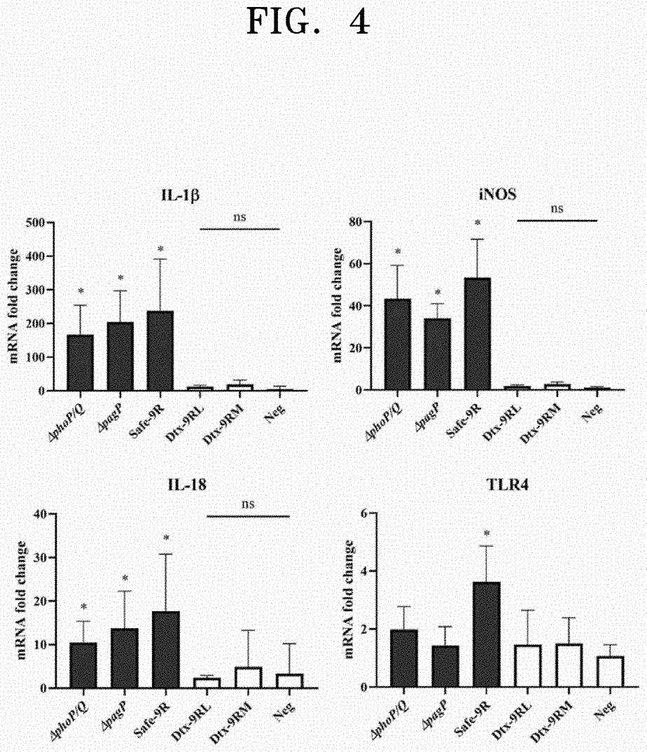

As shown in FIG. 4, cytokine expression levels of the endotoxin-detoxified mutant strains ΔIpxL(Dtx-9RL) and ΔIpxM(Dtx-9RM) were found not to be significantly different from that of the negative control group. As a result, it was found that endotoxins of Dtx-9RL and Dtx-9RM were successfully removed, and did not induce the transcription of IL-16, IL-18, iNOS and TLR4, thereby confirming the potential of Dtx-9RL and Dtx-9RM as endotoxin-detoxified vaccine candidates.

In order to evaluate attenuation of the toxicity of the endotoxin-detoxified mutant strains, immunocompromised chickens (PEM models) were vaccinated therewith and subjected to fasting condition for three days 2 weeks after the vaccination, and an autopsy was performed to identify lesions in the liver and bacterial re-isolation. The results are shown in Table 4.

TABLE 4

| | Group | | | | |
|---|---|---|---|---|---|
| | Dtx-9RL | Dtx-9RM | Safe-9R | SG9R | Negative |
| Lesion | 0/5 | 5/5 | 5/5 | 5/5 | 0/5 |
| Re-isolation | 0/5 | 0/5 | 4/5 | 3/5 | 0/5 |

As shown in Table 4, bacteria were not re-isolated in the endotoxin-detoxified strains and in the Dtx-9RL group, no lesion was observed either. On the other hand, in case of an un-detoxified strain, moderate to severe lesions and bacterial re-isolation were observed.

Next, the effect of the attenuation of the endotoxin-detoxified vaccine strain on the body weight increase was evaluated. Specifically, one-week-old and two-week-old chicks were inoculated with OE Dtx-9RL, OE Dtx-9RM, and OE Safe-9R killed vaccines, and body weights were measured every week for 2 weeks.

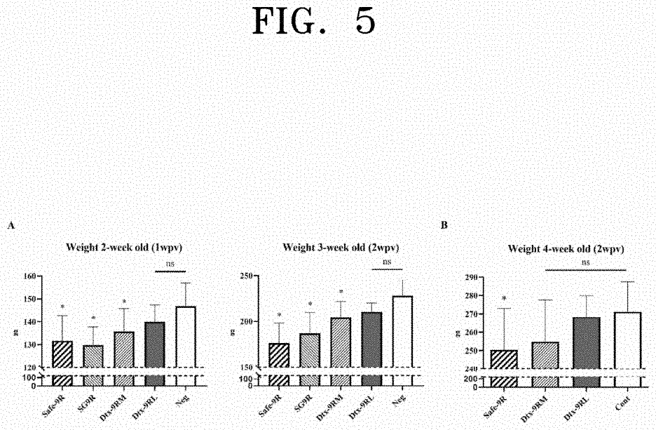

As shown in FIG. 5, upon comparing the body weight increase for two weeks after inoculating to one-week-old chicks, the Dtx-9RL group showed no difference in the body weight compared to the unvaccinated negative control group (FIG. 5A). In addition, upon comparing the body weight increase for two weeks after inoculating to two-week-old chicks, both the Dtx-9RL and Dtx-9RM groups did not show any statistically significant difference, while in case of the un-detoxified strain, reduction in body weight was found (FIG. 5B).

### Example 7. Evaluation of Humoral Immunogenicity of Endotoxin-Detoxified Mutant Strains

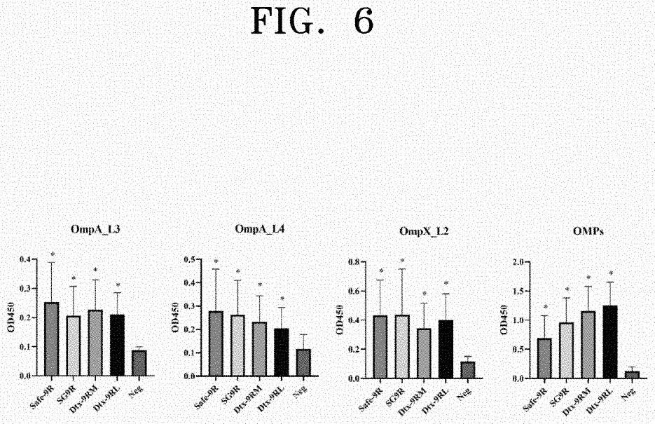

Safe-9R, endotoxin-detoxified mutant strains Dtx-9RL, Dtx-9RM, and SG9R, an existing vaccine strain were inoculated in a form of oil emulsion, antibody titers were measured after 2 weeks, and the results are shown in FIG. 6. Specifically, each vaccine strain was prepared in a form of oil emulsion in the same manner as the OE Safe-9R killed vaccine was produced in Example 5, and each vaccine strain was injected into 15 one-week-old brown layer chicks via the intramuscular route, and blood samples were collected 2 weeks after the vaccination. ELISA was performed in the same manner as in Example 5 to measure the antibody titers.

As shown in FIG. 6, antibody titers were found to be significantly increased in all the vaccinated groups compared to the negative control group.

### Example 8. Evaluation of Protective Efficacy of Live Endotoxin-Detoxified Mutant Strain

One-week-old chicks were inoculated with the live vaccines and challenged after 1 week, and lesions in the liver and bacterial re-isolation were evaluated. The results are shown in Tables 5 and 6.

Firstly, as shown in Table 5, as a result of evaluating protective efficacy of the live endotoxin-detoxified mutant strains, Dtx-9RM had excellent protective efficacy and showed mild lesions compared to other groups. DTX-9RL group did not show any difference from the negative control group in terms of the mortality rate.

TABLE 5

| | Group | | | | | | | | | |
|---|---|---|---|---|---|---|---|---|---|---|
| Vaccinated | Dtx-9RL | | Dtx-9RM | | Safe-9R | | SG9R | | Negative | |
| Age | A ¹ | B ² | A | B | A | B | A | B | A | B |
| 0 ^{a} | 7 | 4 | 2 | 2 | 2 | 2 | 3 | 2 | 4 | 5 |
| 5 | 1 | 0 | 8 | 4 | 0 | 1 | 1 | 1 | 0 | 1 |
| 2 | 0 | 1 | 0 | 3 | 3 | 5 | 5 | 3 | 2 | 0 |
| 3 | 1 | 1 | 0 | 1 | 5 | 2 | 1 | 4 | 2 | 1 |
| 4 | 1 | 4 | 0 | 0 | 0 | 0 | 0 | 0 | 2 | 3 |
| Severe liver lesions ^{b} | 20% | 60% | 0% | 40% | 80% | 70% | 60% | 70% | 60% | 40% |
| Number of chickens | 10 | 10 | 10 | 10 | 10 | 10 | 10 | 10 | 10 | 10 |

1 Vaccination at one-week-old and challenged at two-week-old (1wpv). 2 Vaccination at one-day-old and challenged at two-week-old (2 wpv).

a Liver lesion scoring is as follows: 0: normal; 1: less than 5 necrotic foci; 2: less than 100 necrotic foci; 3: highly multiple (countless) necrotic foci and severe hepatomegaly

b Proportion of liver lesion score 2 or more

Next, after the challenge of the live endotoxin-detoxified mutant strain, the bacteria were re-isolated. The reisolates were identified as, a smooth colony or a rough colony. As a result, as shown in Table 6, bacterial re-isolation was identified in all the vaccinated groups except the Dtx-9RM group, when challenged at 1 wpv. The isolated bacteria were all a smooth strain, that is, the challenge strain. When challenged at 2 wpv, bacterial reisolation was not observed in all the vaccinated groups except SG9R, and the reisolated bacteria were all a rough strain, that is, the vaccine strain. As a result, it was found that when chicks are infected with fowl typhoid at 1 wpv, the existing vaccine (SG9R) does not provide sufficient protective efficacy, and the pathogenic field strain becomes dominant, but when chicks are vaccinated with a endotoxin-detoxified mutant strain, in particular, Dtx-9RM, immunity is quickly established compared to when vaccinated with the existing vaccine strain.

TABLE 6

| | 1 wpv ^{a} challenge | | | | | 2 wpv challenge | | | | |
|---|---|---|---|---|---|---|---|---|---|---|
| | Group | | | | | | | | | |
| | Dtx- | Dtx- | Safe- | | | Dtx- | Dtx- | Safe- | | |
| | 9RL | 9RM | 9R | SG9R | Negative | 9RL | 9RM | 9R | SG9R | Negative |
| Re- | 2/9 ^{b} | 0/10 | 4/10 | 3/10 | 4/8 ^{b} | 0/6 ^{b} | 0/10 | 0/10 | 1/10 | 0/7 ^{b} |
| isolation | | | | | | | | | | |
| Smooth/ | 0/0 | —^{e} | 10/0 | 5/0 ^{d} | 10/0 | — | — | — | 0/4 ^{d} | |
| Rough ^{c} | | | | | | | | | | |

a wpv: week post-vaccination. b Bacteria re-isolation was not performed in deceased chickens.

c Plate agglutination test was performed with Salmonella anti-0 antigen antiserum.

d Maximally 10 and all colonies were tested.

e Colony was not formed.

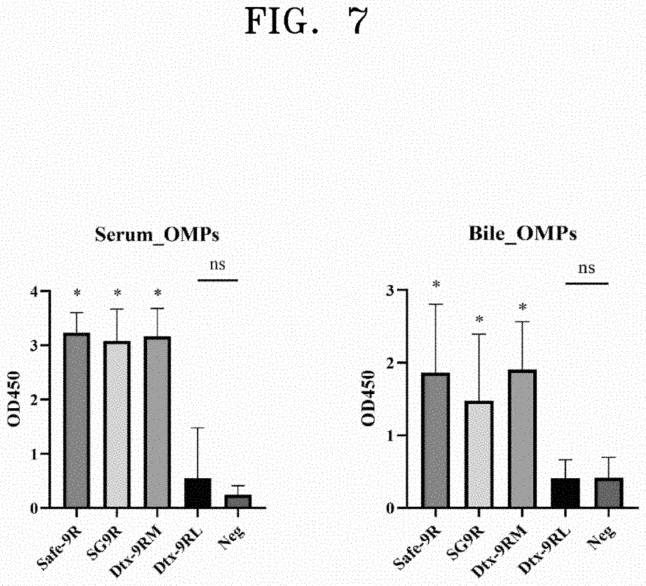

Additionally, humoral and mucosal immunogenicity of Safe-9R, and endotoxin-detoxified mutant strains Dtx-9RL and Dtx-9RM were compared with that of the existing vaccine strain SG9R. Specifically, one-week-old chicks were vaccinated and challenged with the virulent field strain SG0197 (Korean J. Vet. Res. 2015, 55, 241-246) after 1 week of vaccination, blood and bile samples were collected 2 weeks after the challenge and analyzed with the ELISA. As a result, as shown in FIG. 7, it was confirmed that all vaccinated groups except the Dtx-9RL group showed high humoral protective efficacy and mucosal immunity stimulation.

### Example 9. Evaluation of Cellular Immunogenicity of Endotoxin-Detoxified Mutant Strains

The proportion of CD8+ T cells in peripheral blood mononuclear cells (PBMCs) was determined by using FACS (fluorescence activated cell sorting). Specifically, one-day-old chicks were inoculated with endotoxin-detoxified mutant strains, and whole blood samples were collected in heparin-containing tubes. The samples were collected by group, PBMCs were isolated by using a Lymphoprep (Axis Shield, Scotland), and then washed with PBS (phosphate buffered saline) supplemented with 2% FBS (fetal bovine serum). PBMCs were counted and adjusted to a density of 106 cells/mL. 3 μL of CD8+ T-cell antibody-FITC (fluorescein isothiocyanate) and CD4 T-cell antibody-APC (allophycocyanin) were inoculated into 50 μL aliquot of cells from each group, and each aliquot was incubated on ice for 15 minutes in the dark. After incubation, the cells were washed and resuspended in 300 μL of PBS, and were analyzed by using a FACSCalibur (Becton Dickinson).

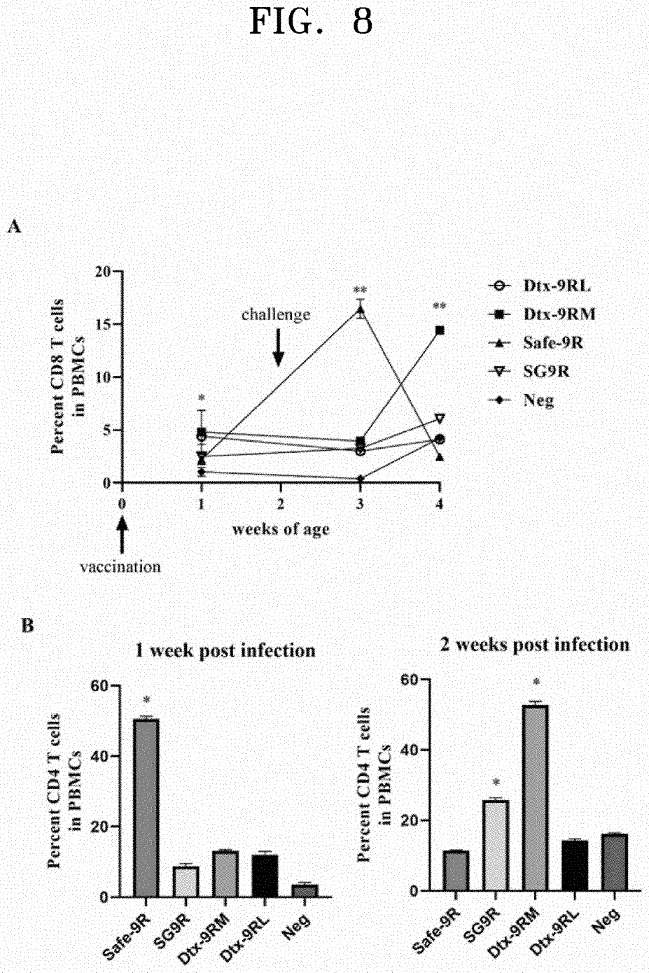

As a result, as shown in FIG. 8, groups vaccinated with endotoxin-detoxified mutant strains at 1 wpv were identified to show a significantly higher percentage of CD8+ T cells than the negative control group. However, the Dtx-9RL group and the negative control group did not exhibit a high T cell percentage, unlike other groups. Therefore, the endotoxin-detoxified vaccine candidate strains are assumed to establish immunity by quickly overcoming the host defense due to their attenuation, and as the Dtx-9RL group did not show any increase in the T cell percentage even after the challenge, the group was confirmed to show the same tendency as identified in the evaluation of the protective efficacy and immunogenicity through the ELISA.

### Example 10. Generation of Vaccine Candidates with Antibiotic Resistance Gene Removed

In the above described Examples, fowl typhoid vaccine candidates Safe-9R and endotoxin-detoxified mutant strains Dtx-9RL and Dtx-9RM were generated to overcome the limitations of the existing SG9R. Additionally, a Dtx-9RM-derived vaccine candidate was generated by using by the FLP-RFT recombination system to remove the antibiotics resistance gene, which was used in selecting vaccine strains.

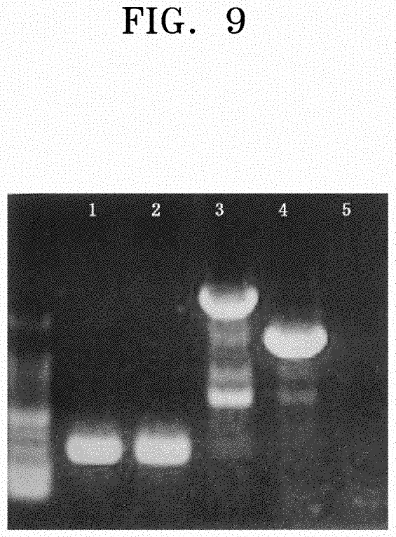

Specifically, in case of Dtx-9RM, in the process of removing the endotoxin by use of by the FLP-RFT recombination system, the target sequence was replaced with an antibiotics resistance gene which is flanked with the FRT regions for later removal by FLP (flippase). The Dtx-9RM has a kanamycin resistance gene and the plasmid having FLP inserted has a tetracycline resistance gene. Each vaccine strain was transformed with the FLP plasmid and cultured overnight on LB-tetra agar at 30° C. for selection. Next, a single colony taken was cultured overnight in LB broth at 37° C. to express FLP, and was spread on both LB agar and LB-kana agar to select the strain in which the kanamycin resistance gene between the FRTs is removed by FLP. As shown in FIG. 9, the bands were found to be blurred or disappear in Dtx9RMΔkana (Lanes 1 and 2), and the antibiotic resistance gene was confirmed to be successfully removed.

The Dtx-9RM Δkana strain was deposited to the Korean Collection for Type Cultures of Korea Research Institute of Bioscience and Biotechnology on May 20, 2021, and was given the accession number of KCTC14577BP.

The above description is only for illustrative purposes, and those skilled in the art to which the present disclosure belongs will be able to understand that the examples and embodiments can be easily modified without changing the principle or essential features of the disclosure. Therefore, it should be understood that the above examples are not limitative, but illustrative in all aspects.

What is claimed is:

1. A Salmonella Gallinarum strain in which an rfaJ and an IpxL genes are deleted.

2. The Salmonella Gallinarum strain of claim 1, wherein the strain is deposited as accession number KCTC14463BP.

3. A Salmonella Gallinarum strain in which an rfaJ gene and an IpxM gene are deleted.

4. The Salmonella Gallinarum strain of claim 3, wherein the strain is deposited as accession number KCTC18822BP.

5. A vaccine composition for preventing fowl typhoid comprising the Salmonella Gallinarum strain of claim 1 as an active ingredient.

6. The vaccine composition of claim 5, wherein the strain is deposited as accession number KCTC14463BP.

7. The vaccine composition of claim 5, wherein the vaccine composition is administered via at least one route selected from the group consisting of oral, percutaneous, intramuscular, intraperitoneal, intradermal, subcutaneous and intranasal routes.

8. The vaccine composition of claim 5, wherein the vaccine composition is injected into any one poultry selected from chickens, ducks, turkeys, geese, quails, pheasants and wild geese.

9. The vaccine composition of claim 8, wherein the poultry is a chick less than 2 weeks old.

10. A vaccine composition for preventing fowl typhoid comprising the Salmonella Gallinarum strain of claim 3 as an active ingredient.

11. The vaccine composition of claim 10, wherein the strain is deposited as accession number KCTC14463BP.

12. The vaccine composition of claim 10, wherein the vaccine composition is administered via at least one route selected from the group consisting of oral, percutaneous, intramuscular, intraperitoneal, intradermal, subcutaneous and intranasal routes.

13. The vaccine composition of claim 10, wherein the vaccine composition is administered to any one poultry selected from chickens, ducks, turkeys, geese, quails, pheasants and wild geese.

14. The vaccine composition of claim 13, wherein the poultry is a chick less than 2 weeks old.
